(19) United States
(12) Patent Application Publication
(10) Pub. No.: US 2024/0379234 A1
(43) Pub. Date: Nov. 14, 2024

(54) QUESTION DECOMPOSITION IN VISUAL QUESTION ANSWERING

(71) Applicant: NEC Laboratories America, Inc., Princeton, NJ (US)

(72) Inventors: Vijay Kumar Baikampady Gopalkrishnan, Santa Clara, CA (US); Samuel Schulter, Long Island City, NY (US); Manmohan Chandraker, Santa Clara, CA (US)

(21) Appl. No.: 18/659,506

(22) Filed: May 9, 2024

Related U.S. Application Data
(60) Provisional application No. 63/465,605, filed on May 11, 2023.
(60) Provisional application No. 63/466,442, filed on May 15, 2023.

(51) Int. Cl. G16H 50/20; G16H 20/00

(52) U.S. Cl. CPC G16H 50/20; G16H 20/00

(57) ABSTRACT

Methods and systems for visual question answering include decomposing an initial question to generate a sub-question. The initial question and an image are applied to a visual question answering model to generate an answer and a confidence score. It is determined that the confidence score is below a threshold value. The sub-question is applied to the visual question answering model, responsive to the determination that the confidence score is below a threshold value, to generate a final answer.

### RELATED APPLICATION INFORMATION

This application claims priority to U.S. Patent Application No. 63/465,605, filed on May 11, 2023, and to U.S. Patent Application No. 63/466,442, filed on May 15, 2023, incorporated herein by reference in their entirety.

### BACKGROUND

### Technical Field

The present invention relates to visual question answering and, more particularly, to the use of task decomposition to improve visual question answering efficiency.

### Description of the Related Art

Visual question answering (VQA) is a machine learning task that incorporates semantic understanding from both textual and graphical sources. An image may be provided to VQA model along with one or more questions. The model then seeks to answer the provided questions using information gleaned from the input image.

However, VQA models tend to consume similar resources regardless of whether the question is simple or difficult. When considering human cognition, certain questions can be answered quickly and without significant effort, while others may need more significant consideration. In contrast, VQA models may apply a same algorithm to every question, resulting in inefficiency when simple questions are considered.

### SUMMARY

A method for visual question answering include decomposing an initial question to generate a sub-question. The initial question and an image are applied to a visual question answering model to generate an answer and a confidence score. It is determined that the confidence score is below a threshold value. The sub-question is applied to the visual question answering model, responsive to the determination that the confidence score is below a threshold value, to generate a final answer.

A system for visual question answering includes a hardware processor and a memory that stores a computer program. When executed by the hardware processor, the computer program causes the hardware processor to decompose an initial question to generate a sub-question, to apply the initial question and an image to a visual question answering model to generate an answer and a confidence score, to determine that the confidence score is below a threshold value, and to apply the sub-question to the visual question answering model, responsive to the determination that the confidence score is below a threshold value, to generate a final answer.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram illustrating selective question decomposition for visual question answering, in accordance with an embodiment of the present invention;

FIG. 2 is a block/flow diagram illustrating the selective use of decomposed sub-questions in visual question answering, in accordance with an embodiment of the present invention;

FIG. 3 is a block diagram illustrating visual question answering with selective decomposition in a healthcare environment, in accordance with an embodiment of the present invention;

FIG. 4 is a block diagram of a computing device that can perform visual question answering with selective decomposition, in accordance with an embodiment of the present invention;

FIG. 5 is a diagram of an exemplary neural network architecture that can be used as part of a visual question answering model, in accordance with an embodiment of the present invention; and

FIG. 6 is a diagram of an exemplary deep neural network architecture that can be used as part of a visual question answering model, in accordance with an embodiment of the present invention.

### DETAILED DESCRIPTION OF PREFERRED EMBODIMENTS

Question decomposition may be used to break an initial question down into simpler tub-tasks that can be answered independently. In the context of visual question answering (VQA) tasks, selective decomposition may be used as a model-agnostic, optimization-free approach to systematically second-guessing model predictions and correcting errors.

This provides improvements to efficiency, as simple questions can be answered with a lower amount of processing power using a relatively small number of sub-questions, while answers to harder questions may be refined using additional sub-questions. Selective decomposition makes it possible to control these refinements by asking additional questions based on a confidence score, where low-confidence answers may be followed by additional refining sub-questions.

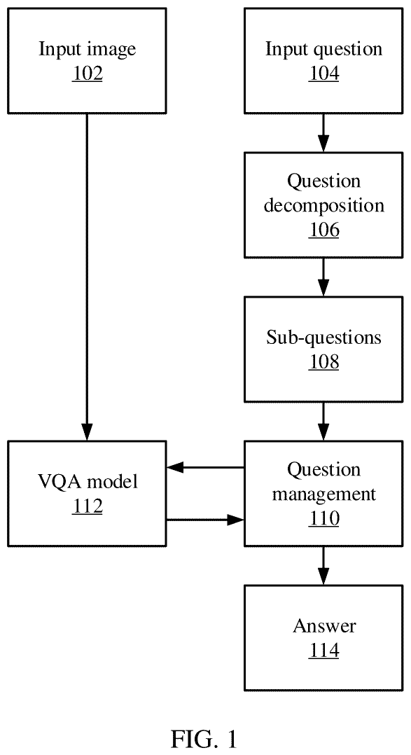

Referring now to FIG. 1, an example of a VQA task with selective question decomposition is shown. The input includes an input image 102 and an input question 104. The input image 102 may depict any appropriate scene, and it is specifically contemplated that the image may include depictions from a medical context. For example, the input image 102 may feature a tissue sample, an image showing a patient with symptoms of a disease, or an image showing posture or movement by a patient with an injury. In another example, the input image 102 may include an aerial photograph showing geographic and man-made features. In another example, the input image 102 may be taken from a security camera, showing a bad actor in the process of committing a crime. The input question 104 asks for information about the image. The information may be identifiable by a visual inspection of the image.

The input image 102 may be applied directly to a VQA model 112, or may undergo its own pre-processing. Before the input question 104 is applied to the VQA model 112, it undergoes question decomposition 106, breaking the input question 104 into a set of sub-questions 108. Question management 110 then applies a first sub-question to the VQA model 112 and receives an answer, along with a confidence score. If the confidence score is below a threshold value, question management 110 may additional sub-questions 108 until it receives an above-threshold confidence score or until a maximum number of sub-questions or processing time has been reached. The question management 110 may then output a final answer 114.

In zero-shot VQA, the model 112 may be expressed as ƒ: ν, q→α, where ν is the input image 102, q is the input question 104, and a is the answer 114. The model ƒ(·) has not been trained on ν, q, α triplets. In practice, this may occur when ƒ(·) is a foundation model that has billions of parameters and has undergone large-scale pre-training for high-level reasoning and knowledge-based abilities. Re-training the model 112 on VQA pairs specifically may be resource-intensive and may damage the robustness of the model. For example, ƒ(·) may be implemented as an autoregressive, generative language model that can optionally be conditioned on the visual modality. Such a model approximates Πₖ₌₁^{N}ρ(tₖ₊₁|t_{1:k}, ν), where ν is an image and t_{1:k} is a sequence of k language tokens. In a zero-shot VQA setting, ƒ(·) understands that it has been giving a question q and should produce a correct answer α in the context of the image ν by modeling it as ρ(α|ν, q).

Question decomposition 106 is the task of decomposing a complex main question into one or more simpler sub-questions 108 that are logically related to the main question 104. Question management 110 uses answers to the sub-questions 108 to compose a final answer 114. This strategy is analogous to how humans approach problem solving.

For example, consider a human who is confronted with a wild animal they have never seen before. The main question in this example may be, “Does this animal pose a threat to me?” The human may decompose this into sub-questions such as, “Does the animal have sharp teeth?” and “Does the animal have forward-facing eyes typical of a predator?” Knowing the answer to even one of these sub-questions makes it much easier to answer the main question.

The task of question decomposition thus includes decomposing a main visual question (ν, q) into one or more sub-questions (s₁, . . . , sₙ). The sub-questions may be answered to obtain decomposition ((s₁, α₁), . . . , (sₙ, αₙ)). Using (ν, q) together with the decomposition, the final answer α is generated.

As noted above, the sub-questions 108 may be generated on the basis of being logically related to the input question 104 and may be simpler than the input question 104. It is difficult to operationalize these concepts to measure whether a given sequence of text is a valid sub-question. A consequentialist view of determining the quality of sub-questions may therefore be applied. The quality of a sub-question may be determined by measuring the effect of the sub-question on reaching an answer. In some cases, sub-question quality may be determined by prompting a pre-trained large language model to determine whether a given sub-question makes sense in view of the input question.

Concretely, (ν, q, α) is defined to be a visual question triplet. Let ρ_{ƒ}(α|ν, q) be the probability of the ground-truth answer α as assessed by a visual question answering model ƒ(·). A decomposition is regarded as being high-quality if ρ_{ƒ}(α|ν, q)<ρ_{ƒ} (α|ν, q, ((s₁, α₁), . . . , (sₙ, αₙ))). In other words, if seeing the decomposition increases the probability of the ground-truth answer α, then it is a quality decomposition. In practice, this criterion may be reduced to determining whether the decomposition induces the model to reproduce the ground-truth answer α.

For a main question triplet (ν, q, α), there is a paired question and answer (q′, α′) for the same image ν such that knowing the answer α to the main question implies the answer α′ to the question q′. For example, given a high-level reasoning question such as, “Can I eat this banana?” a model that says “yes” should also reply “yellow” to the low-level perceptual question of, “What is the color of the banana?” Low-level perception questions and answers relate to identifiable visual characteristics of the image and may be used as an oracular decomposition for the high-level reasoning question, as low-level perception questions are simpler than high-level reasoning questions. In-context learning may be used to perform the VQA task, making use of a prompt that has the main visual question (ν, q) along with a human-written oracular sub-question and sub-answer (s₁, s_{α}_{₁}) for the main question (ν, q).

A decomposition model g(·) performs decomposition as d_{g} (ν, q)→q′. The sub-question q′ may be answered by the VQA model 112 as ƒ(ν, q′)=α′ to produce the sub-question/answer pair (q′, α′). The effectiveness of the decomposition may be measured by the error correction rate:

$$\frac{{\sum }_{i = 1}^{N} 1 [ f ( {v}_{i} , {q}_{i} ) \neq {a}_{i} ∧ f ( {v}_{i} , {q}_{i} ( {q}_{i}^{′} , {a}_{i}^{′} ) ) = {a}_{i} ]}{{\sum }_{i = 1}^{N} 1 [ f ( {v}_{i} , {q}_{i} ) \neq {a}_{i} ]}$$

where (νᵢ, qᵢ, αᵢ) represents the it image, question, and ground-truth answer, respectively, and (qᵢ′, αᵢ′) represents a sub-question generated by the decomposer model and an answer predicted for the sub-question, respectively. The indicator function 1[·] is equal to 1 when the input condition is true and is equal to 0 otherwise. An error induction rate may be considered instead, which measures how often the produced decompositions flipped an answer that was initially correct to an incorrect answer.

The error rate measures the number of instances on which ƒ(·) initially predicted a wrong answer, but switched to the correct answer after seeing the decomposition generated by g(·). This can be understood as the effectiveness of a decomposer model at correcting the errors of the VQA model. In some cases, the same pre-trained model can be used for both ƒ(·) and g(·) with different prompts being used to trigger the different parts of the task.

In a realistic scenario, where it is not known a priori what the answer to the question is, applying sub-questions runs the risk of flipping an initially correct answer to an incorrect answer. To address this second-guessing problem, the initial answer is kept, without further sub-questions when the answer has a high degree of confidence.

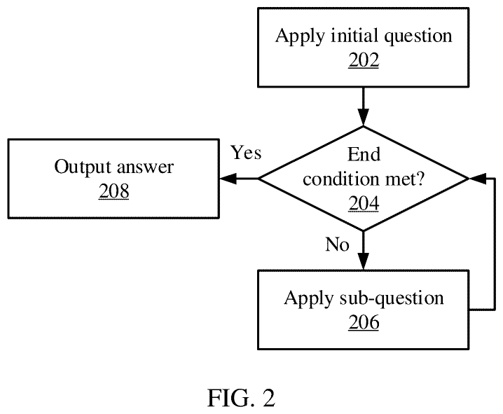

Referring now to FIG. 2, a method of selective decomposition is shown. The selective decomposition is performed by question management 110, which determines whether to ask further sub-questions. Block 202 applies the initial question q to the VQA model 112 and determines an initial answer. Block 204 determines whether an end condition has been met. For example, the end condition may include determining that a maximum number of sub-questions have been asked. If so, the current answer is output in block 208. A second end condition may include a comparison of a confidence score a, output by the VQA model 112 with the initial answer, to a threshold value τ.

If the confidence value is below the threshold value τ, then block 206 applies a sub-question q′ to the VQA model 112. This generates a sub-answer α′ and a corresponding updated confidence score. The updated confidence may be determined on the basis of the original question and the sub-question (e.g., \hat{α}=ƒ(ν, q, q′, α′)). For example, a large language model may provide a score for the output between zero and one to indicate the likelihood of the corresponding word, given the input question. Processing then returns to block 204 to determine whether an end condition has been met, based on the new confidence score.

In some embodiments, the maximum number of sub-questions may be 1, so that only a single sub-question is asked. In some embodiments, any appropriate number of sub-questions may set as the maximum. The threshold value may be specified as a hyper-parameter. There is a wide range of threshold values τ that improve predictive accuracy. The value of τ may be determined empirically or may be selected using validation sets to determine a value that provides the best results. VQA tends to benefit more from selective decomposition when working with input images that are non-naturalistic or from specialized domains than in domains that include natural images. This reflects the intuitive understanding that second-guessing an answer is a better choice in domains that the model has less exposure to.

When applying a question or sub-question to the VQA model 112, a prompt is generated that provides information to the VQA model 112. The information may include a task description and an exemplar. For example, a prompt may be formulated as:
exemplar=“Context: Is the sky blue? No. Are there clouds in the sky? Yes. Question: What weather is likely? Short answer: Rain.”
prompt=exemplar+“Context: {subquestion}? {subanswer}. Question: {question}? Short answer:”

These are in-context examples. The model can understand the general task (e.g., generating the sub-question) from these exemplary demonstrations, making it possible for the model to infer the specific structure and style needed to perform the task on a given input. A human being may generate these exemplar demonstrations to configure the model for sub-question generation, but human input is not needed afterward to guide the model's generation of sub-questions.

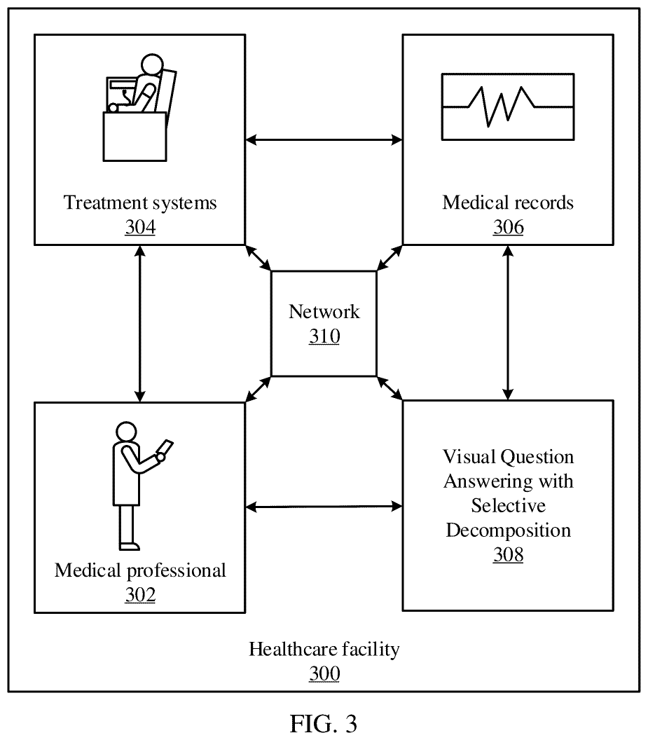

Referring now to FIG. 3, a diagram of information extraction is shown in the context of a healthcare facility 300. Visual question answering with selective decomposition 308 may be used to help identify features of a tissue sample image. For example, such an image may include cancerous tissue from a tumor. In an example, the VQA model may be asked questions about what kind of cancer is shown in the image. The selective decomposition may break that question down into sub-questions such as, “What is the proportion of tumor cells to healthy cells,” or, “Do the cells show a structure typical of this organ?” The visual question answering with selective decomposition 308 may draw the image from medical records 306, and in some cases the questions and sub-questions may further be influenced by information about the patient in the medical records 306.

The healthcare facility may include one or more medical professionals 302 who review information extracted from a patient's medical records 306 to determine their healthcare and treatment needs. These medical records 306 may include self-reported information from the patient, test results, and notes by healthcare personnel made to the patient's file. Treatment systems 304 may furthermore monitor patient status to generate medical records 306 and may be designed to automatically administer and adjust treatments as needed.

Based on information drawn from the visual question answering with selective decomposition 308, the medical professionals 302 may then make medical decisions about patient healthcare suited to the patient's needs. For example, the medical professionals 302 may make a diagnosis of the patient's health condition and may prescribe particular medications, surgeries, and/or therapies.

The different elements of the healthcare facility 300 may communicate with one another via a network 310, for example using any appropriate wired or wireless communications protocol and medium. Thus visual question answering with selective decomposition 308 receives images and questions from medical professionals 302, from treatment systems 304, from medical records 306, and updates the medical records 306 with the output of the VQA model, and further may coordinate with treatment systems 304 in some cases to automatically administer or alter a treatment. For example, if the visual question answering with selective decomposition 308 indicates a dangerous health condition, the treatment systems 304 may automatically alter or halt the administration of the treatment.

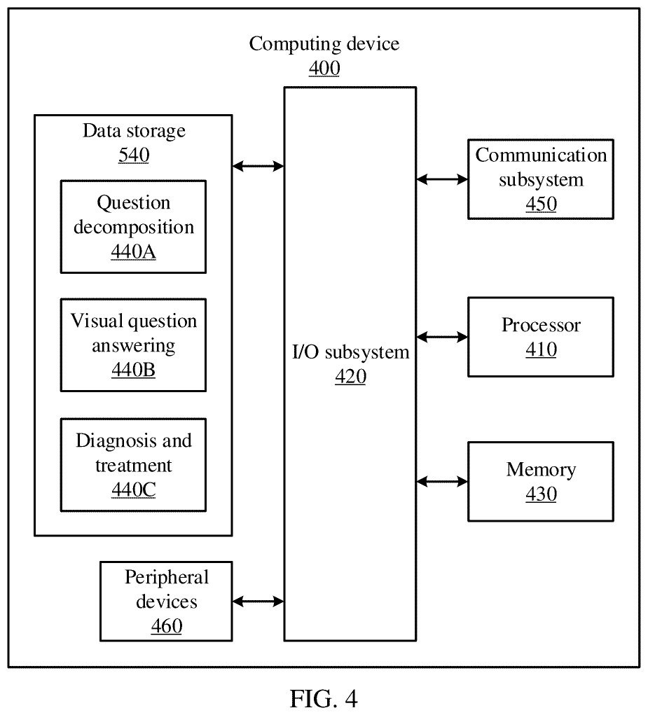

Referring now to FIG. 4, an exemplary computing device 400 is shown, in accordance with an embodiment of the present invention. The computing device 400 is configured to perform visual question answering.

The computing device 400 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 400 may be embodied as one or more compute sleds, memory sleds, or other racks, sleds, computing chassis, or other components of a physically disaggregated computing device.

As shown in FIG. 4, the computing device 400 illustratively includes the processor 410, an input/output subsystem 420, a memory 430, a data storage device 440, and a communication subsystem 450, and/or other components and devices commonly found in a server or similar computing device. The computing device 400 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 430, or portions thereof, may be incorporated in the processor 410 in some embodiments.

The processor 410 may be embodied as any type of processor capable of performing the functions described herein. The processor 410 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 430 may be embodied as any type of volatile or non-volatile memory or data storage capable of performing the functions described herein. In operation, the memory 430 may store various data and software used during operation of the computing device 400, such as operating systems, applications, programs, libraries, and drivers. The memory 430 is communicatively coupled to the processor 410 via the I/O subsystem 420, which may be embodied as circuitry and/or components to facilitate input/output operations with the processor 410, the memory 430, and other components of the computing device 400. For example, the I/O subsystem 420 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 420 may form a portion of a system-on-a-chip (SOC) and be incorporated, along with the processor 410, the memory 430, and other components of the computing device 400, on a single integrated circuit chip.

The data storage device 440 may be embodied as any type of device or devices configured for short-term or long-term storage of data such as, for example, memory devices and circuits, memory cards, hard disk drives, solid state drives, or other data storage devices. The data storage device 440 can store program code 440A for question decomposition, 440B for visual question answering, and/or 440C for performing diagnosis and treatment. Any or all of these program code blocks may be included in a given computing system. The communication subsystem 450 of the computing device 400 may be embodied as any network interface controller or other communication circuit, device, or collection thereof, capable of enabling communications between the computing device 400 and other remote devices over a network. The communication subsystem 450 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 400 may also include one or more peripheral devices 460. The peripheral devices 460 may include any number of additional input/output devices, interface devices, and/or other peripheral devices. For example, in some embodiments, the peripheral devices 460 may include a display, touch screen, graphics circuitry, keyboard, mouse, speaker system, microphone, network interface, and/or other input/output devices, interface devices, and/or peripheral devices.

Of course, the computing device 400 may also include other elements (not shown), as readily contemplated by one of skill in the art, as well as omit certain elements. For example, various other sensors, input devices, and/or output devices can be included in computing device 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 400 are readily contemplated by one of ordinary skill in the art given the teachings of the present invention provided herein.

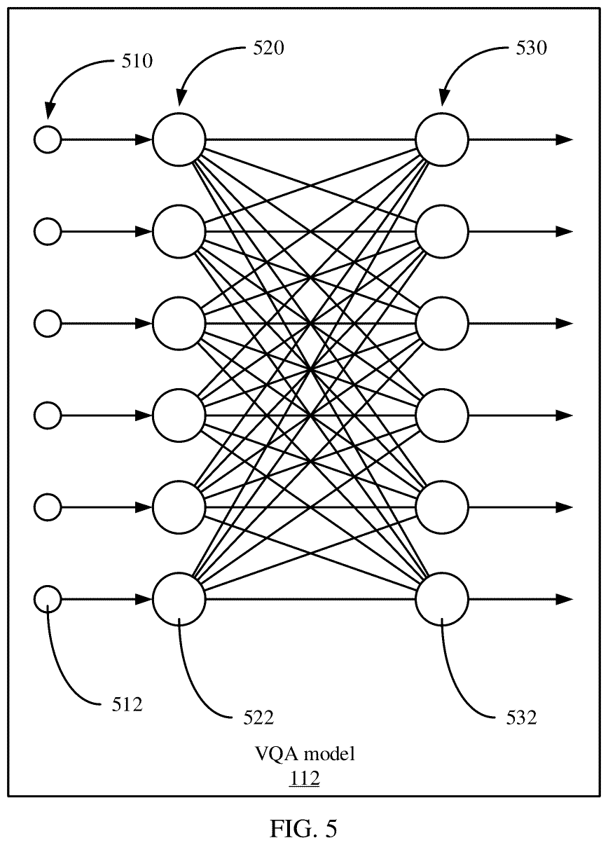

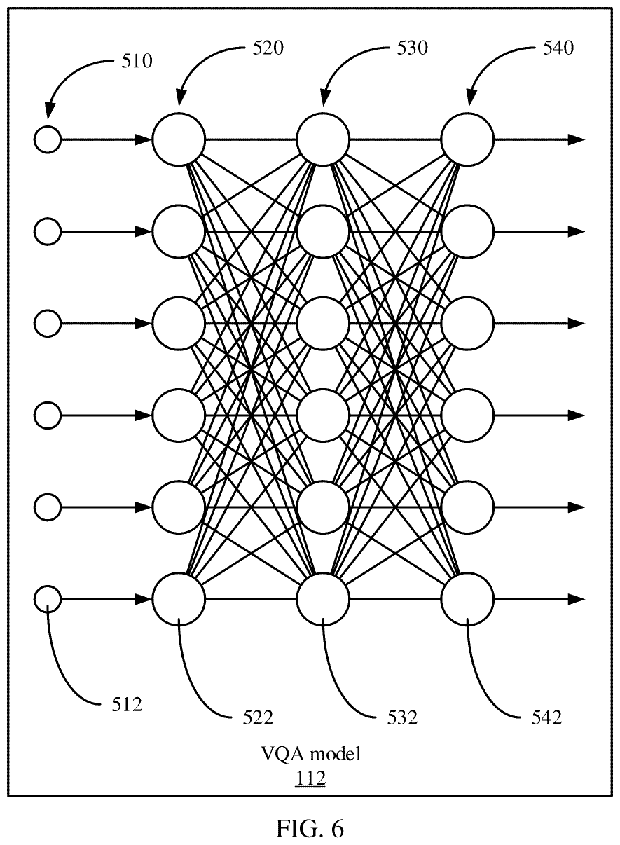

Referring now to FIGS. 5 and 6, exemplary neural network architectures are shown, which may be used to implement parts of the present machine learning models, such as the VQA model 112. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 520 of source nodes 522, and a single computation layer 530 having one or more computation nodes 532 that also act as output nodes, where there is a single computation node 532 for each possible category into which the input example could be classified. An input layer 520 can have a number of source nodes 522 equal to the number of data values 512 in the input data 510. The data values 512 in the input data 510 can be represented as a column vector. Each computation node 532 in the computation layer 530 generates a linear combination of weighted values from the input data 510 fed into input nodes 520, and applies a non-linear activation function that is differentiable to the sum. The exemplary simple neural network can perform classification on linearly separable examples (e.g., patterns).

A deep neural network, such as a multilayer perceptron, can have an input layer 520 of source nodes 522, one or more computation layer(s) 530 having one or more computation nodes 532, and an output layer 540, where there is a single output node 542 for each possible category into which the input example could be classified. An input layer 520 can have a number of source nodes 522 equal to the number of data values 512 in the input data 510. The computation nodes 532 in the computation layer(s) 530 can also be referred to as hidden layers, because they are between the source nodes 522 and output node(s) 542 and are not directly observed. Each node 532, 542 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 532 in the one or more computation (hidden) layer(s) 530 perform a nonlinear transformation on the input data 512 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for visual question answering, comprising:
decomposing an initial question to generate a sub-question;
applying the initial question and an image to a visual question answering model to generate an answer and a confidence score;
determining that the confidence score is below a threshold value; and
applying the sub-question to the visual question answering model, responsive to the determination that the confidence score is below a threshold value, to generate a final answer.

2. The method of claim 1, wherein decomposing the initial question includes applying the initial question to a decomposition model to generate a perception question relating to the initial question.

3. The method of claim 1, wherein applying the sub-question to the visual question answering model further generates a new confidence score for the final answer and wherein decomposing the initial question generates a plurality of sub-questions.

4. The method of claim 3, further comprising iteratively applying sub-questions to the visual question answering model until the new confidence score exceeds the threshold value.

5. The method of claim 1, further comprising performing an action responsive to the final answer.

6. The method of claim 5, wherein the image is an image of a patient and the final answer relates to diagnosis of a medical condition of the patient.

7. The method of claim 6, wherein the action includes automatic administration of a treatment to the patient on the basis of the diagnosis.

8. The method of claim 6, wherein the action includes assistance to medical decision making by healthcare personnel.

9. The method of claim 1, further comprising selecting the threshold value based on a domain of the initial question and the image.

10. The method of claim 1, wherein the visual question model is a machine learning model.

11. A system for visual question answering, comprising:
a hardware processor; and
a memory that stores a computer program which, when executed by the hardware processor, causes the hardware processor to:
decompose an initial question to generate a sub-question;
apply the initial question and an image to a visual question answering model to generate an answer and a confidence score;
determine that the confidence score is below a threshold value; and
apply the sub-question to the visual question answering model, responsive to the determination that the confidence score is below a threshold value, to generate a final answer.

12. The system of claim 11, wherein the computer program further causes the hardware processor to apply the initial question to a decomposition model to generate a perception question relating to the initial question.

13. The system of claim 11, wherein the computer program further causes the hardware processor to generate a new confidence score for the final answer and wherein decomposing the initial question generates a plurality of sub-questions.

14. The system of claim 13, wherein the computer program further causes the hardware processor to iteratively apply sub-questions to the visual question answering model until the new confidence score exceeds the threshold value.

15. The system of claim 11, wherein the computer program further causes the hardware processor to perform an action responsive to the final answer.

16. The system of claim 15, wherein the image is an image of a patient and the final answer relates to diagnosis of a medical condition of the patient.

17. The system of claim 16, wherein the action includes automatic administration of a treatment to the patient on the basis of the diagnosis.

18. The system of claim 16, wherein the action includes assistance to medical decision making by healthcare personnel.

19. The system of claim 11, wherein the computer program further causes the hardware processor to select the threshold value based on a domain of the initial question and the image.

20. The system of claim 11, wherein the visual question model is a machine learning model.
